SEQUENCE LISTING (2 sequences)

SEQ ID NO: 1 - DNA, 23 bp, Artificial Sequence
  Synthetic
tcctccgctttggcgcctcttcc

SEQ ID NO: 2 - DNA, 23 bp, Artificial Sequence
  Synthetic
tgggggttgcaggggagatcctg

(19) United States
(12) Patent Application Publication
(10) Pub. No.: US 2017/0058349 A1
(43) Pub. Date: Mar. 2, 2017

(54) METHODS FOR MANAGING CARE OF PATIENTS PREDISPOSED TO PROGRESSIVE MITRAL VALVE DISEASES

(71) Applicants: The Children's Hospital of Philadelphia, Philadelphia, PA (US); The Trustees of the University of Pennsylvania, Philadelphia, PA (US)

(72) Inventors: Robert J. LEVY, Marion Station, PA (US); Giovanni FERRARI, Philadelphia, PA (US)

(73) Assignees: The Children's Hospital of Philadelphia, Philadelphia, PA (US); The Trustees of the University of Pennsylvania, Philadelphia, PA (US)

(21) Appl. No.: 15/123,321

(22) PCT Filed: Mar. 3, 2015
(86) PCT No.: PCT/US2015/018497

§ 371 Date: Sep. 2, 2016

Related U.S. Application Data
(60) Provisional application No. 61/947,684, filed on Mar. 4, 2014.

(51) Int. Cl. C12Q 1/68; A61K 38/17; G01N 33/94

(52) U.S. Cl. CPC C12Q 1/6883; G01N 33/942; G01N 33/9406; A61K 38/1709; C12Q 2600/106; C12Q 2600/156; C12Q 2600/16; G01N 2800/326

(57) ABSTRACT

The present invention relates to methods for treating or preventing a mitral valve disease in a subject in need thereof, comprising administering to the subject an effective amount of a therapeutic agent. The therapeutic agent is capable of suppressing serotonin receptor signaling. The methods may be combined with a mitral valve surgery, serotonin transporter polymorphism genotyping, MV disease diagnosis, and/or an adjunct assay. Also provided are related medicaments, pharmaceutical compositions, and methods for preparing the medicaments.

### CROSS-REFERENCE TO RELATED APPLICATION

This application claims the benefit of U.S. Provisional Application No. 61/947,684, filed Mar. 4, 2014, the contents of which are incorporated herein by reference in their entireties for all purposes.

### FIELD OF THE INVENTION

The invention relates generally to diagnosis and treatment of mitral valve diseases.

### BACKGROUND OF THE INVENTION

Myxomatous mitral valve disease (MMVD) is one of the leading indications for surgical valve replacement/repair in the US. Mitral valve (MV) disease includes a large spectrum of cardiovascular conditions, such as myxomatous mitral valve disease (MMVD) and chronic ischemic mitral regurgitation (MR) among others, and can only be treated surgically. MV prolapse is defined as a single or bileaflet prolapse, at least 2 mm above the annular plane in the long-axis view, with or without leaflet thickening on echocardiography. MMVD occurs in approximately 7.2 million individuals in the US and over 144 million worldwide, and is therefore a critically important clinical problem. Currently, no diagnostic or therapeutic treatments exist for the risk-stratification or treatment of these groups of patients, with surgical intervention (Mitral Valve Repair or Replacement) as the only available options. Patients are followed by echocardiographic analysis till a symptom occurs, then require surgery.

Myxomatous degeneration is defined by the accumulation of mucopolysaccharides responsible for the thickening and “proliferative” aspect of the valve tissue. Increasing evidence suggests that mitral valve interstitial cells (MVICs) play a critical role in the pathological remodeling of the MV leaflets.

The serotonin transporter (SERT or 5HTT) polymorphism is a 43 base pair DNA sequence that has been shown to present in full-length form (LL) in 25% of human populations, heterozygous in 50% (LS), and deleted in 25% (SS). Some reports indicate a 44 base pair deletion/insertion. It was first published by Lesch at al. (Science. 1996 Nov. 29; 274(5292):1527-31), and has been of interest to the fields of psychiatry and neuroscience, chiefly because of the importance of SERT as a target for the treatment of depression. SERT-polymorphism genotype has been used for diagnosing predisposition to depression and other mental disorders.

There remains a need for diagnosis and pharmacotherapies for subjects who are predisposed to progressive mitral valve diseases such as myxomatous mitral valve disease (MMVD).

### SUMMARY OF THE INVENTION

The present invention relates to treatment and/or prevention of mitral valve diseases in subjects who are predisposed to progressive mitral valve diseases.

A method for treating or preventing a mitral valve disease in a subject in need thereof is provided. The method comprises administering to the subject an effective amount of a therapeutic agent, which is capable of suppressing serotonin receptor signaling. According to this method, the signaling activity of a serotonin receptor in the subject may be suppressed; the metabolism of serotonin in the subject may be modified; the progression of the mitral valve disease in the subject may be retarded; activation of mitral valve interstitial cells in the subject may be reversed; activation of mitral valve endothelial cells in the subject may be reversed; and/or mitral valve remodeling in the subject may be reversed.

The mitral valve disease may be a myxomatous mitral valve disease.

The serotonin receptor may be selected from the group consisting of 5HTR2A and 5HTR2B.

The therapeutic agent may be selected from the group consisting of serotonin receptor inhibitors, serotonin transporter inhibitors, monamine oxidase inhibitors and anti-oxidants.

The subject may have LL serotonin transporter polymorphism. The subject may not receive a serotonin release drug. The subject may suffer from the mitral valve disease and receive a mitral valve surgery. The mitral valve surgery may be selected from the group consisting of mitral valve repair and mitral valve replacement with a prosthesis.

The method may further comprise determining the serotonin transporter polymorphism in the subject. The serotonin transporter polymorphism determination may comprise performing a genotyping assay on a nucleic acid sample comprising a serotonin transporter gene promoter from the subject. The genotyping assay may comprise (a) amplifying a portion of the serotonin transporter gene promoter; and (b) determining whether the serotonin transporter gene promoter is in an LL form.

The method may further comprise diagnosing the subject as having the mitral valve disease. The mitral valve disease diagnosis may comprise clinical examination of the subject. The mitral valve disease diagnosis may comprise documentation of the mitral valve disease in the subject using an imaging technique. The imaging technique may be selected from the group consisting of cardiac ultrasound, magnetic resonance imaging and cardiac catheterization.

The method may further comprise measuring the blood level of a serotonin transporter gene related biomarker in the subject. The biomarker may be selected from the group consisting of serotonin, 5-hydroxyindolacetic acid, and a catecholamine.

### BRIEF DESCRIPTION OF THE DRAWINGS

FIGS. 1A-F show human microarray analysis and identification of 5HT signaling pathways in MMVD patients. (A) Heat maps showing the relative expression levels of genes from the MMVD and control samples (N=4). Ordering of the genes and samples is based on a hierarchical clustering and the expression values across rows are Z-score normalized for visualization purposes only. (B) (C) and (D) Tables showing selected genes differentially expressed between MMVD and Controls for 5HT, ECM, and TGF-81 signaling, respectively. Fold changes and p value are indicated. (E) and (F): Heat maps showing the relative expression levels of genes from the MMVD and control expression samples for the TGFB signaling pathway (hsa04350) or the serotonin pathways (hsa04726).

FIGS. 2A-D show that human mitral valve leaflet remodeling is associated with increased expression of 5HTR2s. (A) Representative H&E staining of 4 μm thick cross sections of human MV leaflets surgically resected from MMVD patients and controls (N=4 group). (B) Modified Movat Pentachrome staining (Magnification 40×). (C) and (D) Immunohistochemistry staining of human MV leaflets using anti-5HTR2A and anti-5HTR2B antibodies, respectively. Magnification, 63×.

FIGS. 3A-D show SERT genetic polymorphisms. DNA was isolated from 254 patients enrolled via the PennCardiacBioregistry according to approved IRB protocols. (A) Schematic representation of SERT genetic polymorphisms. (B) Distribution of SERT polymorphisms in patients with type I MV disease (ischemic MVP) (C) Distribution of SERT polymorphisms in patients with type II MV disease (Myxomatous MVP) (D) Frequency of LL polymorphism in patients with type II MV disease (Myxomatous MVP) organized by age groups.

FIG. 4 shows MVIC activation in vitro. MVICs isolated from healthy controls and patients with LL, LS, SS gene polymorphisms were treated with 10 mM 5HT for 6 days in the presence or absence of a combination of 5HT2A antagonist Ketanserin, 5HT2B antagonist SDZ, and SERT antagonist Fluoxetine. RNA was isolated and tested for SMA expression as a marker of MVIC activation.

FIGS. 5A-D show that angiotensin II infusion provokes remodeling of the mitral valve tissue in mice. (A) Representative H&E staining of cross section of mice hearts harvested 28 days after saline or AngII chronic infusion. (B) Modified Movat Pentachrome staining of MV leaflets harvested 28 days after saline or AngII chronic infusion (Magnification 10× and 43×). (C) and (D) Immunohistochemistry staining of murine MV leaflets using anti-5HTR2A and anti-5HTR2B antibodies, respectively.

FIGS. 6A-G show that ischemic mitral regurgitation (IMR) in an ovine model is associated with 5HTR2 overexpression. (A) Schematic representation of left ventricle for chronic IMR. (B) Echo of MV regurgitation before and after an ischemic event, myocardial infarct (MI). (C) Excised left ventricle with infarct and MV. (D) Representative H&E analysis of ovine MV leaflets at baseline, 1, 4 and 8 weeks post-MI. Magnification 4×. (E) Modified Movat Pentachrome staining of ovine MV leaflets harvested at baseline, 1, 4 and 8 weeks post MI. (F) and (G) Representative immunohistochemistry staining of ovine MV leaflets using anti-5HTR2A and anti-5HTR2B antibodies, respectively.

FIG. 7 shows gel electrophoresis of DNA fragments obtained by PCR for identification of LL (512 bp) polymorphism, SS (469 bp) polymorphism, or LS polymorphism. 3% agarose gel was used. PCR program: 95° C.—15 min; 94° C.—30 sec; 65.5° C.—90 sec; 72° C.—60 sec; 35 cycles from step 2; 72° C.—10 min.

FIG. 8 shows a snapshot of Peak Scanner analysis for identification of SERT polymorphisms.

### DETAILED DESCRIPTION OF THE INVENTION

The present invention relates to care management for subjects who suffer from or are predisposed to mitral valve diseases, including progressive mitral valve diseases. The invention is based on the discovery of an enhanced frequency of a long serotonin transporter (SERT) polymorphism in the promoter region of the SERT gene over the expected Mendelian's distribution in myxomatous mitral valve disease (MMVD) patients requiring a cardiac surgery, and the discovery of a combination of the serotonin polymorphism genotyping with cardiac diagnostic imaging techniques to determine if a patient is predisposed to a progressive mitral valve (MV) disease such as MMVD. The invention is also based on the discovery of a link between serotonin receptor (5HTR) signaling and MMVD. MMVD has been found to be associated with increased serotonin receptor (5HTR) signaling, which enhances downstream processing of serotonin (5HT) resulting in increased reactive oxygen species (ROS) activities. The SERT-polymorphism test of the present invention provides several major advantages over the current approach to MMVD, including identifying a population at risk for more rapid progression; identifying potential risks involved in this population if they were also taking serotonin-related drugs; and providing guidance for future clinical trials to identify serotonin-related medications that could be beneficial for treating MMVD medically, for example, postponing or avoiding surgery intervention.

In particular, using microarray analysis and a network reconstruction, a link between 5HT and MMVD in human has been confirmed. Genotyping 253 surgical patients with different MV conditions has revealed an enhanced frequency of the SERT-LL polymorphism over the expected Mendelian's distribution. Notably the highest frequency was among the youngest subjects (53.4% vs. the expected 25%) (p=0.009) in patients under 60 years of age. Histology confirms altered expression of 5HTR-2A, -2B, and SERT in MMVD. In vitro assays on human MVICs show that VIC activation is associated with 5HTR activation and that antagonists of 5HTR2A (Ketanserin), 2B (SDZ), and SERT (Fluoxetine) partially abrogate this effect. Patient with LL polymorphism are more prone to MVICs activation in vitro, supporting the idea that these subgroups of patients are at a higher risk of 5HT-mediated valvulopaties. Using ovine and murine models 5HT-mediated remolding of the MV has been confirmed: C57BL/6J mice were treated with Angiotensin II to induce MV remodeling with results showing increased (p<0.01) cusp thicknesses, ECM remodeling, and increased 5HTR-2B/SERT expressions in AngII-treated mice vs. saline. Pathological remodeling of the MV leaflets was induced using an ovine model of ischemic mitral regurgitation with results showing 5HT signaling along ECM remodeling and MVICs activation.

According to the present invention, SERT-genotyping provides a novel means of characterizing patients with MMVD into a subgroup with an increased risk for rapid progression. Furthermore, MMVP patients may benefit from a pharmacotherapy that can alter 5HT-related mechanisms.

The present invention provides methods for managing care of subjects who suffer from or are predisposed to mitral valve (MV) diseases, especially progressive MV diseases such as MMVD. The methods may comprise treating or preventing an MV disease in a patient by 5HTR antagonism and/or inhibition of related mechanistic downstream events, such as serotonin transporter (SERT) activity and oxidative stress, and alteration (e.g., retardation) of the progression of pathological prolapse of a mitral valve leaflet. The present invention also provides a novel method for identifying MMVD patients with an increased risk for rapid progression of the disease, who may benefit from a pharmacotherapy with medications that can alter serotonin-related mechanisms. The present invention further provides a combination of SERT-genotyping with MMVD diagnosis.

A method for treating or preventing a mitral valve (MV) disease in a subject in need thereof is provided. The method comprises administering to the subject an effective amount of a therapeutic agent that is capable of suppressing serotonin receptor (5HTR) signaling.

The MV disease may be myxomatous mitral valve disease (MMVD) or chronic ischemic mitral regurgitation (MR), preferably the MV disease is MMVD. The MV disease may be progressive.

The term “progressive” as used herein is defined as worsening of an MV disease with increasing symptoms, which may be physical limitations such as exercise impairment, chest pain, shortness of breath, and cardiac arrhythmias. A progressive mitral valve disease may be a myxomatous mitral valve disease (MMVD) (sometimes called Barlow′ syndrome) or other less common types of mitral valve diseases such as congenital malformations of the mitral valve, rheumatic fever related mitral valve diseases, or mitral valve diseases due to a coronary artery disease with an ischemic injury to the mitral valve apparatus.

The subject may be an animal, including a mammal, for example, a human, a mouse, a cow, a horse, a chicken, a dog, a cat, a sheep, and a rabbit. The animal may be an agricultural animal (e.g., horse, cow, sheep and chicken) or a pet (e.g., dog and cat). The subject is preferably a human, a sheep or a mouse, more preferably a human. The subject may be a male or female. The subject may also be a newborn, a child or an adult. The subject may be of any age. For example, the subject may be a human under about 70, 65, 60, or 55 years old, preferably under about 65 years old.

The subject may suffer from an MV disease or may be predisposed to an MV disease. Preferably, the subject suffers from an MV disease, but has not exhibited one or more symptoms of a progressive MV disease.

The subject may have LL serotonin transporter (SERT) polymorphism. Human serotonin transporter (SERT) gene transcription is modulated by a common polymorphism in its upstream regulatory region. There are long and short variants of this promoter region. The long form (L) has the full length of the promoter region while the short form (S) has a deletion of a 43 base pair DNA sequence. LL SERT polymorphism refers to a genotype having two copies of the long form (L) of the promoter allele of the SERT gene. SS SERT polymorphism refers to a genotype having two copies of the short form (S) of the promoter allele of the SERT gene. LS SERT polymorphism refers to a genotype having one copy of the long form (L) and one copy of the short form (S) of the promoter allele of the SERT gene.

A subject having LL SERT polymorphism may have an increased risk for developing a progressive MV disease than a subject not having LL SERT polymorphism. The term “risk” as used herein refers to a time dependent increase in probability of consequences due to progressive valve disease, and these consequences include death, stroke, disability, risks associated with cardiac catheterization, catheter intervention and open heart surgery, including risks of death and disability due to catheterization (cath) or surgical intervention. The risk may be significant if there is a numerical increase by at least, for example, about 1%, 5%, 10%, 20%, 30%, 40%, 50%, 60%, 70%, 80%, 90%, 95%, or 100° A), preferably by at least about 10%, more preferably by at least about 50%, most preferably by at least about 100%, over a predetermined period of time (e.g., 1, 3, 6, 12, 18, 24, 30, 36 or 60 months). A significant risk may or may not be of statistical significance. In some preferred embodiments, the subject suffers from an MV disease and has LL SERT polymorphism.

The signaling activity of a serotonin receptor in the subject may be suppressed. The serotonin receptor may be any of the 5HT receptor families, for example, 5HTR1, 5HTR2, 5HTR3, 5HTR4, 5HTR6, and 5HTR7, preferably 5HTR2A or 5HTR2B. The 7 general serotonin receptor classes include a total of 14 known serotonin receptors (i.e., 5HTR1A, 5HTR1B, 5HTR1D, 5HTR1E, 5HTR1F, 5HTR2A, 5HTR2B, 5HTR2C, 5HTR5A, 5HTR5B). The suppression may be by at least about 1%, 5%, 10%, 20%, 30%, 40%, 50%, 60%, 70%, 80%, 90%, 95%, or 100%, preferably by at least about 10%, more preferably by at least about 50%, most preferably by at least about 100%, over a predetermined period of time (e.g., 1, 2, 3, 5, 7, 14, 21, 28 or 30 days, or 1, 3, 6, 12, 18, 24, 30, 36 or 60 months) in the treated subject as compared with an untreated control subject.

The serotonin metabolism in the subject may be modified. The modification may be monitored by measuring parameters such as serotonin levels, 5-hydroxyindolacetic acid, or both.

The progression of the MV disease in the subject may be retarded. The retardation may be slowing down or prevention, preferably prevention, of the MV disease. The progression or development of the mitral valve disease may be retarded by, for example, at least about 1%, 5%, 10%, 20%, 30%, 40%, 50%, 60%, 70%, 80%, 90%, 95%, or 100%, preferably at least about 10%, more preferably at least about 50%, most preferably at least about 100%, over a predetermined period of time (e.g., 1, 2, 3, 5, 7, 14, 21, 28 or 30 days, or 1, 3, 6, 12, 18, 24, 30, 36 or 60 months) in the treated subject when compared with an untreated control subject.

In some embodiments, activation of mitral valve interstitial cells (MVICs) in the treated subject is reversed or prevented. Activation of MVICs may be evidenced by the presence of a chondro-osteogenic marker (e.g., RUNX2+, SMA+, OPN+, BMP4+, CRTAC1+) or an elevated level of SMAα2 or SM22α transcript.

In other embodiments, activation of mitral valve endothelial cells (VECs) in the treated subject is reversed or prevented. At present, there is no way to assess VEC activation in a living human subject. However, cell culture techniques could be applied to valve tissue explanted from the subject at surgery. Activation of VECs in cell culture may be detected using various assays. For example, markers such as VEGF+, vWF+, FLK1+, FLT1+, CD31+ may be used to detect VECs activation. A migration assay is a functional test to determine VEC mobility. As endothelial cells are often in a quiescent state, the replication rate of VEC may be used to monitor when VECs re-enter cell cycle once stimulated by, for example, physiological or pathological stimuli.

In yet other embodiments, mitral valve (MV) remodeling in the treated subject is reversed or prevented. The reversal may be evidenced by the expression of an extracellular matrix (ECM) protein (e.g., matrix metalloproteinases (MMPs), tissue inhibitors of metalloproteinases (TIMPs), and glycosaminoglycans (GAG)). The therapeutic agent may be a chemical compound, a biological molecule or a combination thereof, which is capable of suppressing serotonin receptor (5HTR) signaling. The suppress may be by at least about 1%, 5%, 10%, 20%, 30%, 40%, 50%, 60%, 70%, 80%, 90%, 95%, or 100%, preferably at least about 10%, more preferably at least about 50%, most preferably at least about 100%. The therapeutic agent may be a protein such as an antibody or a nucleic acid such as a small interfering RNA (siRNA). The therapeutic agent may be selected from the group consisting of serotonin receptor (5HTR) inhibitors, serotonin transporter (SERT) inhibitors, monamine oxidase inhibitors and anti-oxidants. The 5HTR inhibitors may include 5HTR antagonists, 5HTR siRNAs and 5HTR antibodies. Exemplary 5HTR antagonists include 5HTR1A antagonists (e.g., BMY 7378 cyanopindolol, iodocyanopindolol, lecozotan, methiothepin, methysergide, NAN-190, nebivolol, nefazodone, WAY-100,135, WAY-100,635, mefway, SB216641 and WAY100635), 5HTR1D antagonists (e.g., GR-127,935 ketanserin, metergoline, methiothepin, rauwolscine, ritanserin, vortioxetine, ziprasidone, BRL15572), 5HTR2A antagonists (e.g., ketanserin and MDL100907, clozapine, olanzapine, quetiapine, risperidone, ziprasidone, aripiprazole, asenapine, amitriptyline, clomipramine, cyproheptadine, eplivanserin, etoperidone, haloperidol, hydroxyzine, iloperidone, methysergide, mianserin, mirtazapine, nefazodone, pimavanserin, pizotifen, ritanserin, trazodone, and yohimbine), 5HTR2B antagonists (e.g., SB204741, SB228357, agomelatine, asenapine, bzp, ketanserin, methysergide, ritanserin, rs-127,445, tegaserod, and yohimbine), 5HTR2C inhibitors (SB242,08478), 5HTR2B&2C antagonists (e.g., SB206553) and 5HTR1B antagonists (e.g., GR55562). Preferably, the 5HTR antagonist is ketanserin or GR55562.

The term “an effective amount” as used herein refers to an amount of a therapeutic agent required to achieve a stated goal (e.g., treatment or prevention of an MV disease, modification of 5HT metabolism, suppression of 5HTR, retardation of the progress of an MV disease, reversal or prevention of activation of mitral valve interstitial cells (MVICs) or mitral valve endothelial cells (VECs), and/or reversal of mitral valve (MV) remodeling). The effective amount of a therapeutic agent may vary depending upon the stated goals, the physical characteristics of the subject, the nature and severity of the MV disease, the existence of related or unrelated medical conditions, the nature of the therapeutic agent, the means of administering to the subject, and the administration route. A specific dose for a given subject may generally be set by the judgment of a physician. The therapeutic agent may be administered to the subject in one or multiple doses. In each dose, the therapeutic agent may be present at about 0.001 mg-10 g, preferably about 0.01-1000 mg, more preferably about 1-500 mg, per kg body weight of the subject.

The subject may receive a serotonin-related pharmaceutical therapy. The term “serotonin-related pharmaceutical therapy” as used herein refers to a therapy affecting the serotonin (5HT) pathway in a subject. The therapy is preferably specific for a genotype or known disease characteristics. A SERT inhibitor (such as Fluoxetine) may be indicated in an LL SERT polymorphism subject. A therapy reducing 5HT receptor signaling may be beneficial. It is preferable to use inhibitors specific for receptors (e.g., 5HTR2A and 5HTR2B) known to be involved in a mitral valve disease.

Potential secondary effects of the serotonin-related pharmaceutical therapy in the subject may be assessed. Preferably, the subject does not receive a serotonin (5HT) release drug. The 5HT release drug may be Fenfluramine.

According to the present invention, the treatment or prevention of an MV disease in a subject with a therapeutic agent may be combined with MV disease diagnosis, mitral valve surgery, SERT-polymorphism genotyping, and/or an adjunct assay.

Where the subject has not been diagnosed to have an MV disease, the method of the present invention may further comprise MV disease diagnosis. In particular, the method may further comprise diagnosing the subject as having the MV disease. The MV disease diagnosis may comprise clinical examination of the subject and/or documentation of the MV disease in the subject using an imaging technique. The imaging technique may be selected from the group consisting of cardiac ultrasound, magnetic resonance imaging and cardiac catheterization.

Where the subject suffers from an MV disease, the method of the present invention may further comprise a mitral valve surgery. In particular, the subject may receive a mitral valve surgery. The mitral valve surgery may be mitral valve repair or mitral valve replacement with a prosthesis. The subject may be treated with a therapeutic agent before or after, preferably before, the mitral valve surgery.

Where the subject has not been diagnosed to have a progressive MV disease, the method of the present invention may further comprise genotyping the subject. In particular, the method may comprise determining the serotonin transporter (SERT) polymorphism in the subject. The SERT polymorphism determination may comprise performing a genotyping assay on a nucleic acid sample comprising a SERT gene promoter from the subject. The nucleic acid sample may be any sample from the subject. Exemplary nucleic acid samples include a bodily fluid sample, a blood sample and a urine sample. The genotyping assay may comprise (a) amplifying a portion of the serotonin transporter (SERT) gene promoter, and (b) determining whether the SERT gene promoter is in an LL form. The amplification may be carried out using an amplification primer pair that distinguishes the long promoter allele from other alleles of the SERT gene. The presence of LL SERT polymorphism in a subject indicates that the subject is predisposed to development of a progressive MV disease, or has an increased risk for developing a progressive MV disease.

The method of the present invention may further comprise performing an adjunct assay on the subject. The adjunct assay may indicate an increased risk of developing a progressive mitral valve disease in the subject. The adjunct assay may comprise measuring the blood level of a biomarker related to serotonin transporter (SERT) gene. The biomarker may be selected from the group consisting of serotonin (5HT), 5-hydroxyindolacetic acid and a catecholamine. 5-hydroxyindolacetic acid for example is the principal metabolite of serotonin after SERT processing by monamine oxidase. Increased 5-hydroxyindolacetic acid may reflect a specific undesirable consequence of LL SERT genotype in a subject.

The method may further comprise optimizing the medical care for the subject. The optimization may be guided and adjusted based upon clinical status and serotonin biomarker levels and changes in these parameters. Biomarkers for a progressive mitral valve disease with an increased risk (e.g., 5-hydroxyindolacetic acid and TGF-β) may be used. Forefront imaging techniques may be used to show increased serotonin receptor presence in mitral valve leaflets. Optimized medical care of a patient with an MV disease may exclude a serotonin (5HT) release drug.

A method for reversing activation of a mitral valve interstitial cell (MVIC) from a subject, activation of a mitral valve endothelial cell (VEC) from a subject, or mitral valve (MV) remodeling in a cell from a subject is also provided. The subject has a mitral valve disease and LL SERT polymorphism. The method comprises administering to the cell an effective amount of a therapeutic agent, which is capable of suppressing signaling activity of a serotonin receptor. The reversal of activation of MVICs may be evidenced by the presence of a chondro-osteogenic marker (e.g., RUNX2+, SMA+, OPN+, BMP4+, CRTAC1+) or an elevated level of SMAα2 or SM22α transcript. Action of VECs may be tested using various assays. For example, markers such as VEGF+, vWF+, FLK1+, FLT1+, CD31+ may be used to test VECs activation. A migration assay is another functional test to determine VEC mobility. As endothelial cells are often in a quiescent state, the replication rate of VEC may be used to monitor when VECs re-enter cell cycle once stimulated (either by physiological or pathological stimuli). Reversal of MV remodeling may be evidenced by the expression of an ECM protein (e.g., matrix metalloproteinases (MMPs), tissue inhibitors of metalloproteinases (TIMPs) and glycosaminoglycans (GAG)). The serotonin receptor (5HTR) may be 5HTR2A or 5HTR2B. The agent may be selected from the group consisting of serotonin receptor (5HTR) inhibitors, serotonin transporter (SERT) inhibitors, monamine oxidase inhibitors and anti-oxidants. Exemplary 5HTR inhibitors include 5HTR antagonists, 5HTR siRNAs, and 5HTR antibodies. Exemplary 5HTR antagonists include 5HTR1A antagonists (e.g., SB216641 and WAY100635), 5HTR1D antagonists (e.g., BRL15572), 5HTR2A antagonists (e.g., Ketanserin and MDL100907), 5HTR2B antagonists (e.g., SB204741 and S8228357), 5HTR2C inhibitors (58242,08478), 5HTR2B&2C antagonists (e.g., S8206553), 5HTR1B antagonists (e.g., GR55562). Preferably, the 5HTR antagonist is ketanserin or GR55562. In some embodiments, serotonin (5HT) metabolism is modified in the cell.

For each treatment or prevention method of the present invention, a pharmaceutical composition for treating or preventing a mitral valve (MV) disease in a subject in needed thereof is provided. The composition comprises an effective amount of a therapeutic agent, which is capable of suppressing signaling activity of a serotonin receptor. The effective amount of the therapeutic agent may be selected to achieve a stated goal (e.g., treatment or prevention of an MV disease, modification of 5HT metabolism, suppression of 5HTR, retardation of the progress of an MV disease, reversal or prevention of activation of mitral valve interstitial cells (MVICs) or mitral valve endothelial cells (VECs), and/or reversal of mitral valve (MV) remodeling). The effective amount of a therapeutic agent may vary depending upon the stated goals, the physical characteristics of the subject, the nature and severity of the MV disease, the existence of related or unrelated medical conditions, the nature of the therapeutic agent, the means of administering to the subject, and the administration route. The pharmaceutical composition may further comprise a pharmaceutically acceptable carrier or diluent. Carriers, diluents and excipients suitable in the pharmaceutical composition are well known in the art.

The pharmaceutical compositions of the present invention may be formulated for oral, sublingual, intranasal, intraocular, rectal, transdermal, mucosal, topical or parenteral administration. Parenteral administration may include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids) injection or infusion, preferably intraperitoneal (i.p.) injection in mouse and intravenous (i.v.) in human. Any device suitable for parenteral injection or infusion of drug formulations may be used for such administration. For example, the pharmaceutical composition may be contained in a sterile pre-filled syringe.

For each treatment or prevention method of the present invention, a medicament useful for treating or preventing a mitral valve (MV) disease in a subject in needed thereof is provided. The medicament comprises an effective amount of a therapeutic agent, which is capable of suppressing signaling activity of a serotonin receptor. The medicament may also be useful for modification of 5HT metabolism, suppression of 5HTR, retardation of the progress of an MV disease, reversal or prevention of activation of mitral valve interstitial cells (MVICs) or mitral valve endothelial cells (VECs), and/or reversal of mitral valve (MV) remodeling in the subject.

For each medicament of the present invention, a method for preparing the medicament of the present invention is provided. The preparation method may comprise admixing a therapeutic agent with a pharmaceutically acceptable carrier or diluent.

The term “about” as used herein, when referring to a measurable value such as an amount, a percentage, and the like, is meant to encompass variations of ±20%, ±10%, more preferably ±5%, even more preferably ±1%, and still more preferably ±0.1% from the specified value, as such variations are appropriate.

### Example 1

### Human Microarray Analysis and Identification of 5HT Signaling Pathways in MMVD Patients

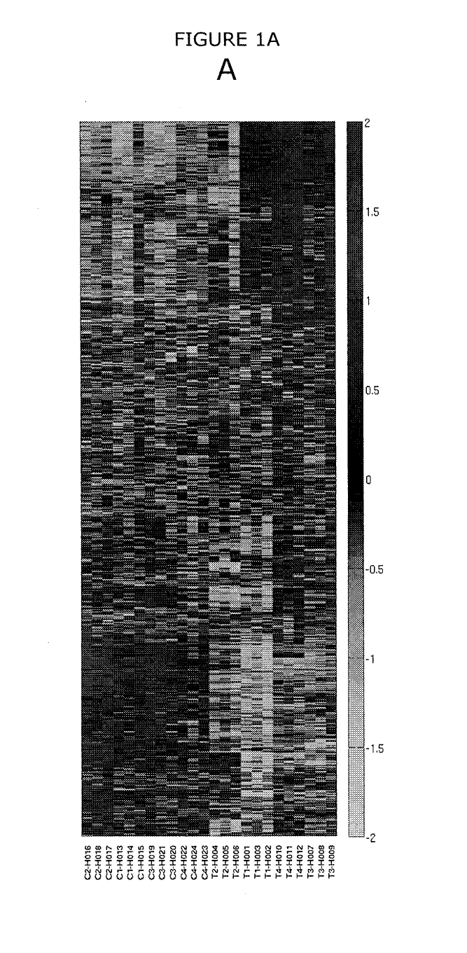

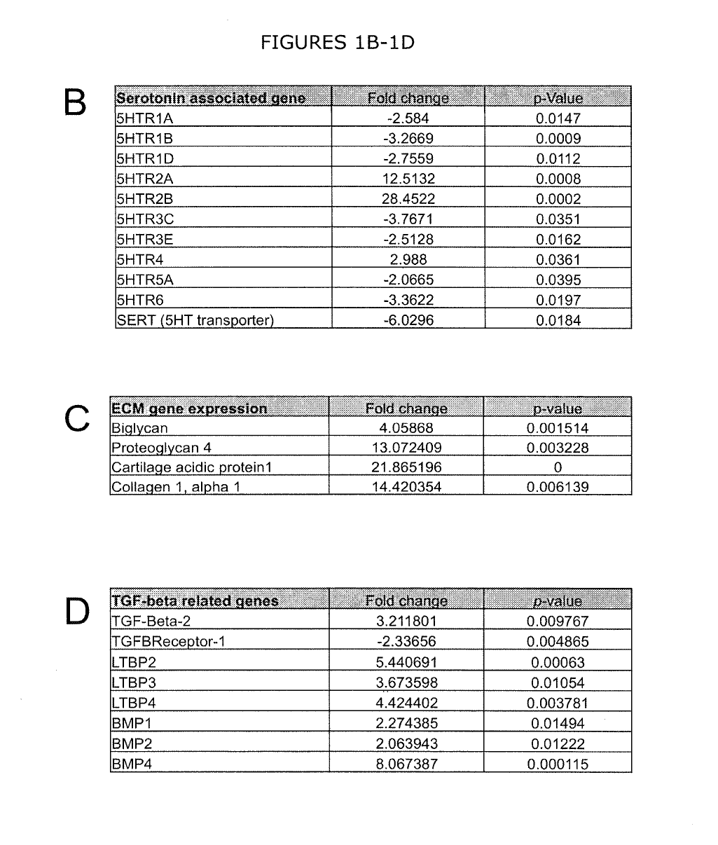

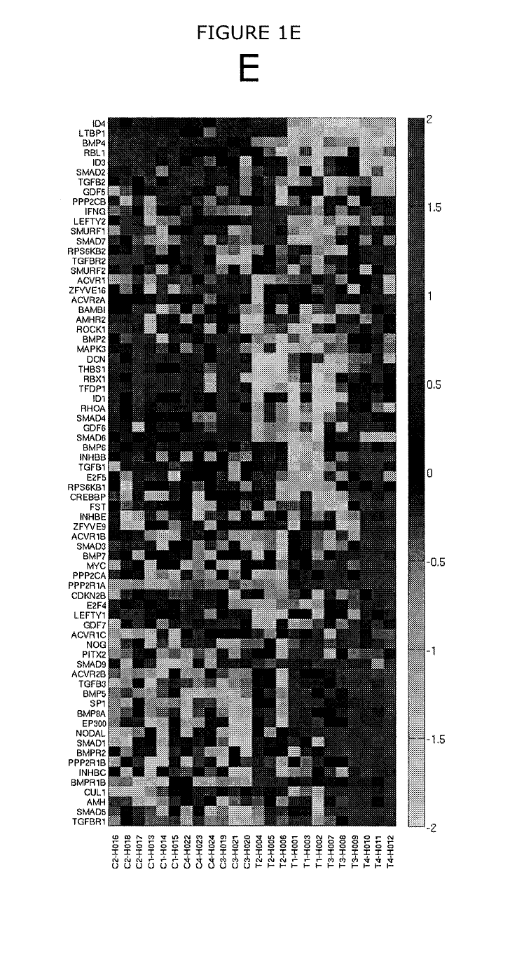

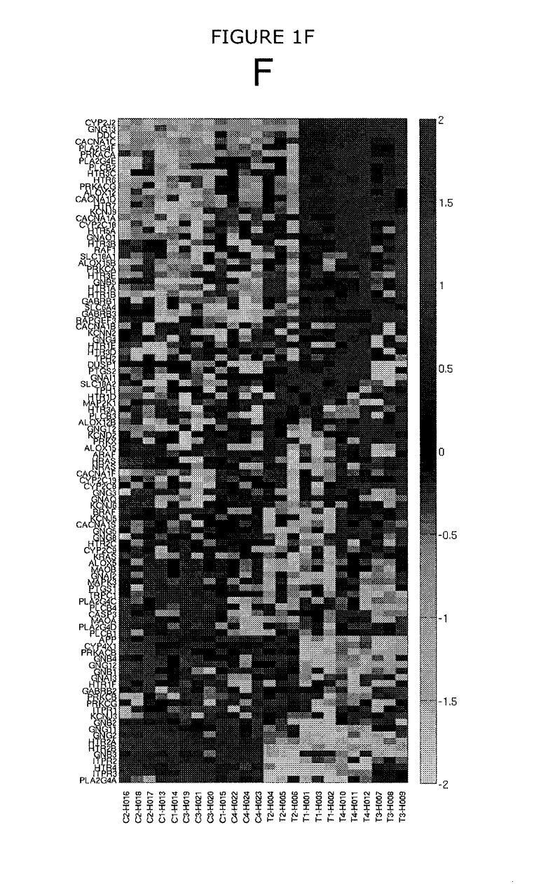

Microarray analysis was performed on samples from 4 MMVD patients and 4 controls. Among the transcriptional activities of 19,553 human sequences determined by use of an oligonucleotide microarray, a total of 1,883 probe sets were found to fulfill the criteria for differential expression (FIG. 1A). These transcripts represent genes showing at least a two-fold change in MMVD tissue vs. controls. Of the 1,883 transcripts considered, 1,033 were upregulated (54.8%) and 850 down regulated (45.2%). Bio-informatics analysis highlighted the differential expression of a number of genes that directly or indirectly indicate the involvement of 5HTR pathways and extracellular matrix remodeling in MMVD (FIGS. 1B-C). Significant increases (12 to 28-fold) were reported in 5HTR2A and 5HTR2B with little to no change in other 5HTRs in the MMVD patients as compared to control. The microarray analysis also confirms differential expression of TGF-beta-related signaling, as a possible regulator of 5HT metabolism (FIGS. 1D-F). These results are in agreement with other observations previously reported concerning 5HTR2 activity and expression in human and canine MVIC. It is of further interest that SERT was significantly down regulated. Furthermore, increased expression of markers of MVIC activation and ECM synthesis (FIG. 1E) was demonstrated for a number of genes including Osteopontin, RUNX2, BMP4, Type 1 collagen, and glycosaminoglycan-associated proteins in agreement with prior results in sheep and porcine aortic VIC, and canine and human MVIC. Thus, 5HT could hypothetically favor progression in MMVD due to increased signaling capacity of 5HTR2A/28, which induces MVIC activation and ECM remodeling. These data suggest a link between human MMVD and 5HT signaling.

### Example 2

### Regulatory Network Reconstruction in MMVD Vs. Controls

PANDA (Passing Attributes between Networks for Data Assimilation) is a message-passing model using multiple sources of information to predict regulatory relationships, and used to integrate protein-protein interaction, gene expression, and sequence motif data to reconstruct genome-wide, condition-specific regulatory networks. First, this method was used to validate a well-known signaling pathway involved in MMVD, TGF-b signaling. It has been reported that the pathological remodeling of the MV is associated with increased level of the TGF-beta-related molecule. The 5HT signaling and the crosstalk between TGF-b1 and 5HT were then analyzed.

The PANDA was applied to integrate gene expression data with transcription-factor motif regulatory information and protein-protein interaction data, constructing two directed, genome-wide regulatory networks, one for the control (C) samples and the other for the MMVD specimens. By comparing the predicted networks, we identified regulatory relationships specific to either the “MMVD” and “C” samples. We identified which of these MMVD-network and C-network regulatory relationships included a member of either the TGF-b signaling pathway or the 5HT pathway. Interestingly, there is a high level of differential-targeting around several important genes in these pathways. For example, TGF-b1 is much more highly targeted (p=2.9e-6) in the control-network with 19 identified specific regulatory interactions, but none in the MMVD network. Among genes belonging to the 5HT pathway, 5HTR2A is more highly targeted in the MMVD-network compared to the control-network (p=4.7e-4) with 8 times as many regulatory relationships. Some of this differential-targeting of pathway genes may be in part mediated by differences in upstream transcription factors. A Venn diagram of the transcription factors targeting TGF-b1 and HTR2A in either the identified C-network or MMVD-network may be prepared. Interestingly, many transcription factors are identified as common regulators of these genes, including E2F proteins. These data are supporting the concept that 5HT signaling is a valuable target for MMVD therapy.

### Example 3

### Human Mitral Valve Leaflet Remodeling is Associated with Increased Expression of 5HTR2s and SERT

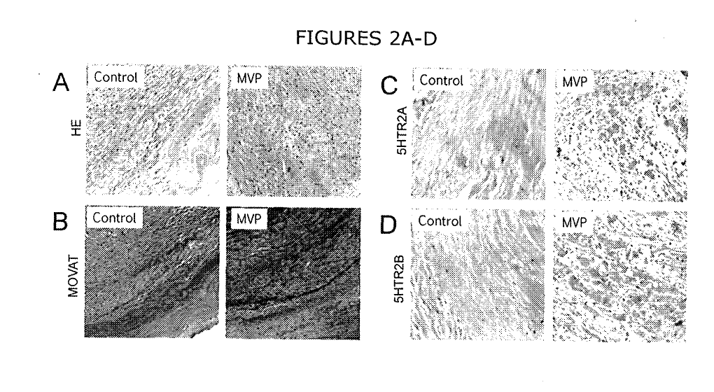

To validate these data, we performed immunohistochemical analysis of surgically resected MV leaflets from control and MMVD patients (N=4/group). FIGS. 2A-B show an increased deposition of collagen and proteoglycan, and elastin disarray in a section of myxomatous mitral valve when compared to control. In addition, the cross section of resected P2 segments shows significant rearrangement of the ECM with expression of ECM proteins such as fibronectin, the proteoglycans (mostly Versican, Lumican, fibromodulin, and biglycan), along with alterations in the type of collagen expression (type I, III and IX). As shown in these experiments, the key microscopic change in MVVD appears to occur in the spongiosa. We then tested whether this pathological remodeling of the tissue in MMVD was associated with overexpression of 5HTR2A and 2B. As shown in FIGS. 2C-D, both 5HTR2A and 2B are over-expressed in MMVD when compared to controls. Notably 5HTR2s are over-expressed throughout the entire MV leaflets. These data confirm that 5HT signaling is altered in vivo.

### Example 4

### Surgical Patients with MMVD have a Higher than Expected Distribution of the Serotonin Transporter (SERT)-LL Polymorphism

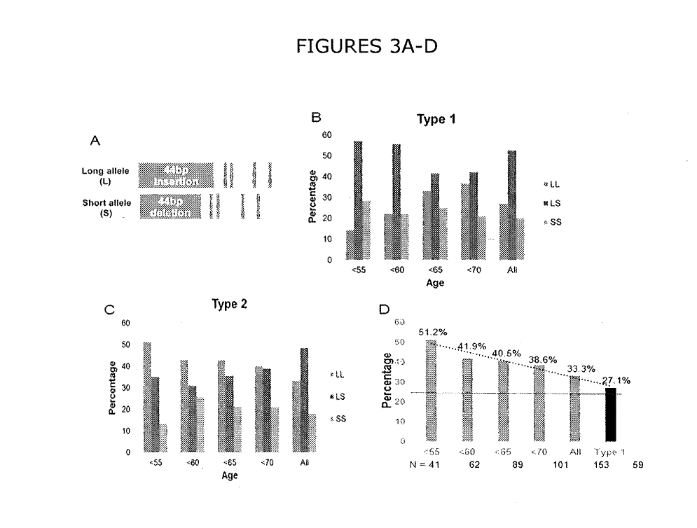

We then characterized the genetic predisposition to MMVD based on Serotonin transporter gene polymorphism. A 44 base polymorphism of a repetitive element in the promoter region of the serotonin transporter (SERT) gene, designated as a short (S) or long (L) allele, has been reported (FIG. 3A), with results showing that the LL form results in an increased maximum velocity of the enzyme-catalyzed reaction for 5HT reuptake compared to the SS form. The echocardiogram and surgical reports of 254 patients undergoing MV surgery were reviewed and three groups were obtained based on MV diagnosis and Carpentier Functional Classification [Type I: Ischemic MVP (N=59), Type II: MMVD (N=152), Type III: rheumatic MVP (N=41)]. DNA was extracted and genomic fragment analysis was performed to determine allelic frequencies. A chi-square statistical test was performed (FIG. 3B). While Type I and Type III patients show the expected polymorphism distribution, patients with MMVD have a higher than expected frequency of LL-SERT polymorphism (34% vs. 25%) (FIG. 3C). Notably, the frequency of SERT-LL is particularly significantly enhanced (53.4% vs. 25%) (p=0.009) in MMVD patients under 60 years old (FIG. 3D).

### Example 5

### 5HTR and SERT Antagonist Partially Reduce MVIC Activation In Vitro

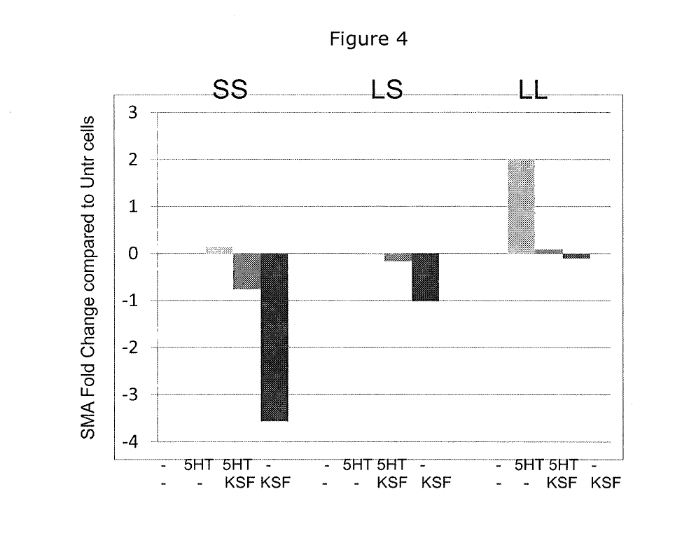

MVICs were isolated form patients with LL, LS, or SS genotype and controls (N=5/group) and exposed to 5HT stimulation (10 nM) to measure MVICs activation by alpha smooth muscle actin (SMA) as previously described by Rachana et al. (Journal of cellular physiology 227(6): 2595-604, November 2011. PMCID: PMC3288540) and Poggio et al. (Cardiovascular research 98(3): 402-10, June 2013. PMCID: PMC3656614). A combination of 5HT2A, 2B and SERT antagonists was used to inhibit 5HT stimulation in the three different genotypes (LL, LS, SS) with 10 μM ketanserin, 1 μM SDZ SER-082, and 1 μM Fluoxitine. (FIG. 4). RNA extract was collected to detect SMA expression. While SS and LS phenotypes were not responsive to 5HT stimulation in this experimental setting, patients with LL showed an significant upregulation of SMA suggesting that these patient were more prone to MVIC activation and therefore MV remodeling than other genotypes. A combination of Ketanserin, SDZ and Fluoxetine reduced 5HT-mediated upregulation of SMA.

### Example 6

### Angiotensin II Infusion Provokes Remodeling of the Mitral Valve Tissue in Mice

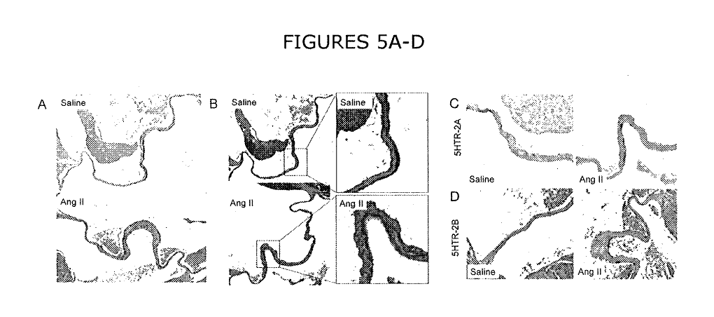

We have recently reported that reactive oxygen species (ROS) accumulation is leading aortic VICs activation ex vivo and in vitro with results showing that adenoviral delivery of antioxidant enzymes ameliorates aortic VICs activation. We therefore used our murine model based on chronic AngII infusion to reproduce ROS accumulation in the cardiac tissue (Branchetti et al., Cardiovasc Res. 2013; 100(2):316-324) to test whether we could provoke MV remodeling. Immunohistological analysis was performed on each animal to test for ECM remodeling and 5HTR2s expression. Microdissection analysis of murine valve tissue shows increased (p<0.01) cusp thickness in AngII treated vs. saline infused mice (FIG. 5A). As per the human study we noticed significant rearrangement of the ECM (FIG. 5B). Histological analysis also revealed increased 5HTR2A and 2B and SERT in MV leaflets (FIGS. 5C-E) as seen in human lesions.

### Example 7

### Ovine Model of Ischemic Mitral Regurgitation is Associated with 5HTR Overexpression

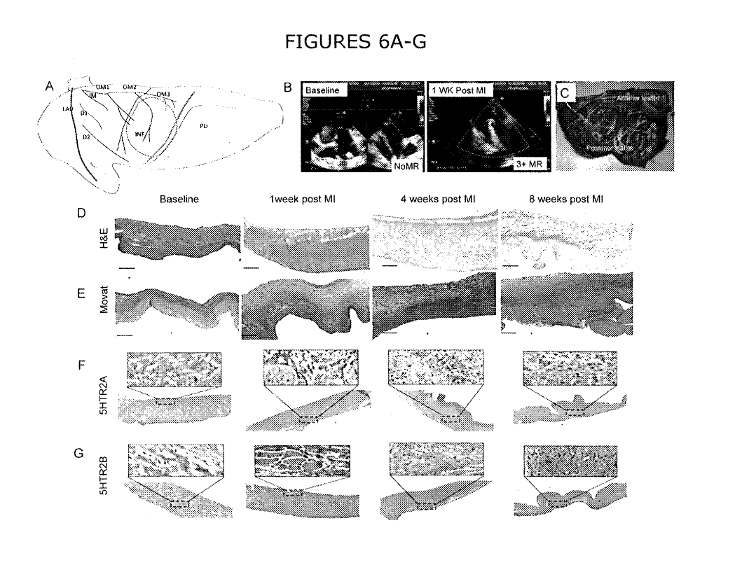

Ischemic MR is a common complication of pathologic remodeling of the left ventricle due to acute and chronic coronary artery diseases. It frequently represents the pathologic consequences of increased tethering forces and reduced coaptation of the MV leaflets. Furthermore it has been associated with remodeling of the MV leaflets. We utilized the Gorman Surgical Laboratory's established ovine myocardial infarct (MI) model to establish MV responses to altered stresses over an 8 week period. Three adult male sheep per time point (N=12) underwent a left thoracotomy to allow ligation of the left circumflex coronary artery branches between the lateral and middle cardiac veins (FIG. 6A). Baseline echocardiogram data was recorded before and after the ligation (FIG. 6B). Animals were euthanized at baseline, 1, 4, and 8 weeks post-MI. The heart was excised, the left ventricle was opened through the interventricular septum, and a digital photograph was taken. MV leaflets were then excised, preserved in formalin, (FIG. 6C) and processed for histological analyses. Notably, starting one-week post-MI we observed increased thickening of the sheep MV leaflets (FIG. 6D), ECM remodeling (FIG. 6E), and overexpression of 5HTR2A/2B (FIG. 6G) in animals presenting with MV regurgitation.

### Example 8

### SERT Genotyping

The inventors explored the possibility that a well-known Mendelian distributed serotonin transporter (SERT) polymorphism in the promoter region of the SERT gene could be associated with an increased risk for more rapidly progressive MMVD, requiring earlier cardiac surgery. Specifically, the SERT-promoter polymorphism is a 43 base pair DNA sequence that has been shown to present in full length form (LL) in 25% of human populations, heterozygous in 50% (LS), and deleted in 25% (SS). The inventors genotyped for SERT-promoter alleles in 124 patients, who required mitral valve surgery. Indications for surgery were based on serial measurements over time of both cardiac imaging data documenting disease progression, declining cardiac function and increased regurgitant fraction, and the clinical presence of increased disabling cardiac symptoms including exercise intolerance, arrhythmias and circulatory collapse.

The results of the inventors studies demonstrated that in this MMVD population requiring surgery, there was a predominance of the LL SERT-polymorphism genotype (as summarized in the data presented in detail below), that was even more apparent when age was taken into account, with an even greater proportion in the under 65y group than those above this age, indicating the need for surgery at an earlier age due to more rapid MMVD disease progression in LL patients.

Patient enrollment. The present investigation conforms to the principles outlined in the Declaration of Helsinki. For the present study, from April 2009 to December 2013, we enrolled a total of 126 subjects, from the Hospital of the University of Pennsylvania and at the Penn Presbyterian Medical Center according to the approved IRB protocol#809349. All the patients had a long history of mitral regurgitation and represented with leaflet thickening, annular dilatation and/or chordal rupture. The surgical technique involved partial resection of the prolapsed P2 segment with placement of a flexible annuloplasty band. There was no residual MR at the time of discharge and there were no surgical complications.

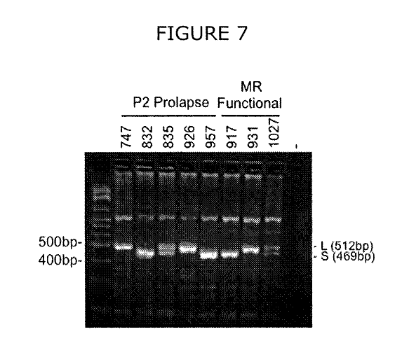

Methods. DNA was isolated from the buffy coat from each individual human subject using the QTAamp Mini Kit (Qiagen). The serotonin transporter gene was amplified by PCR using Platinum Pfx DNA polymerase (Invitrogen Life Technologies) and specific primers (IDT). Samples were then run on a 3% agarose gel. The amplified long form (L) is detected at 512 bp and the short form (S) is detected at 469 bp. (FIG. 7).

Forward:
(SEQ ID NO: 1)
TCCTCCGCTTTGGCGCCTCTTCC
Reverse:
(SEQ ID NO: 2)
TGGGGGTTGCAGGGGAGATCCTG

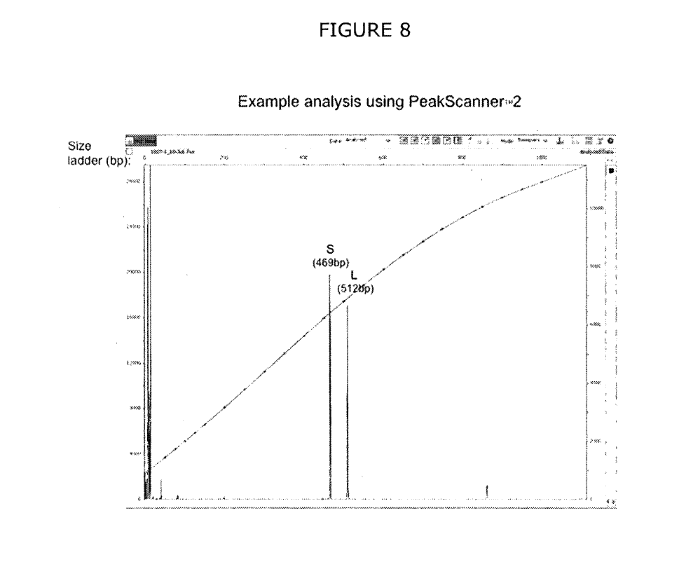

In a second set of experiments to detect the presence of the SERT polymorphism by Fragment Analysis, the serotonin transporter gene was amplified by PCR using Platinum Pfx DNA polymerase (Life Technologies) and specific primers (IDT), with the forward primer synthesized with a 5′ fluorescent label. Dilutions of the PCR amplification were then loaded into a semi-skirted 96-well plate and given to the NAPCORE facility at Children's Hospital of Philadelphia. Fragment analysis was performed using ABI 3730 and results were analyzed using Peak Scanner™ 2 software (Life Technologies). (FIG. 8).

Forward:
(SEQ ID NO: 1)
/56-FAM/-TCCTCCGCTTTGGCGCCTCTTCC
Reverse:
(SEQ ID NO: 2)
TGGGGGTTGCAGGGGAGATCCTG

MMVD patients show a higher than expected frequency of LL-SERT. We first analyzed the presence of SERT polymorphisms in patients presenting with MMVD, other MV pathologies, and in a control population. (Table 1). We analyzed 9 controls, 64 MMVD patients presenting with a prolapse of the P2 segment of the MV, 11 pts presenting with bileaflet prolapse, 34 patients presenting with functional Mitral regurgitation secondary to ischemic events, and 15 patients presenting with other MV pathologies. As presented in Table 1, MMVD patients presented a frequency of LL-SERT of 33% instead of the expected 25%, and 14% of SS-SERT instead of 25%. Non-MMVD patients and control present the expected Mendelian distribution

Patients presenting for surgery at younger age show a higher predominance of LL SERT then older patients. We then analyzed the presence of SERT polymorphisms in the same pts population considering the age of patients at the time of surgery. (Table 2). Our sub analysis, Table 2, shows patients presenting for surgery at younger age (<65 year of age) show even higher frequency of the LL polymorphisms (43% vs the expected 25%) then overall MMVD subgroup.

TABLE 1
SERT Genotyping in MMVD patients

| | LL | | LS | | SS | | |
|---|---|---|---|---|---|---|---|
| Total MV | 36 | (21%) | 64 | (52%) | 23 | (18%) | total 123 |
| expected | 30.75 | (25%) | 61.5 | (50%) | 30.75 | (25%) | |
| Prolapse | 25 | (33%) | 35 | (48%) | 14 | (19%) | total 74 |
| expected | 18.5 | (25%) | 37 | (50%) | 18.5 | (25%) | |
| Func- | 13 | (23%) | 33 | (56%) | 12 | (20%) | total 58 |
| tional, | | | | | | | |
| control, | | | | | | | |
| other | | | | | | | |
| expected | 14.5 | (25%) | 29 | (50%) | 14.5 | (25%) | |

TABLE 2
SERT Genotyping in MMVD patients subgroups

| | LL | | LS | | SS | | |
|---|---|---|---|---|---|---|---|
| CONTROL | 2 | | 4 | | 3 | | total 9 |
| P2 < 65yo | 15 | (43%) | 13 | (37%) | 7 | (20%) | total 35 |
| expected | 8.75 | (25%) | 17.5 | (50%) | 8.75 | (25%) | |
| P2 > 65yo | 7 | (24%) | 17 | (59%) | 5 | (17%) | total 29 |
| expected | 7.25 | (25%) | 14.5 | (50%) | 7.25 | (25%) | |
| Bileaflet | 3 | (27.25%) | 5 | (45.5%) | 3 | (27.25%) | total 11 |
| expected | 2.75 | (25%) | 5.5 | (50%) | 2.75 | (25%) | |
| Functional | 6 | (17.5%) | 23 | (67.5%) | 5 | (15%) | total 34 |
| expected | 8.5 | (25%) | 17 | (50%) | 8.5 | (25%) | |
| Other | 5 | (33.3%) | 6 | (40%) | 4 | (26.7%) | total 15 |
| expected | 3.75 | (25%) | 7.5 | (50%) | 3.75 | (25%) | |

What is claimed:

1. A method for treating or preventing a mitral valve disease in a subject in need thereof, comprising administering to the subject an effective amount of a therapeutic agent, wherein the therapeutic agent is capable of suppressing serotonin receptor signaling.

2. The method of claim 1, whereby the signaling activity of a serotonin receptor in the subject is suppressed.

3. The method of claim 1, whereby the metabolism of serotonin in the subject is modified.

4. The method of claim 1, whereby the progression of the mitral valve disease in the subject is retarded.

5. The method of claim 1, whereby activation of mitral valve interstitial cells in the subject is reversed.

6. The method of claim 1, whereby activation of mitral valve endothelial cells in the subject is reversed.

7. The method of claim 1, whereby mitral valve remodeling in the subject is reversed.

8. The method of claim 1, wherein the subject has LL serotonin transporter polymorphism.

9. The method of claim 2, wherein the serotonin receptor is selected from the group consisting of 5HTR2A and 5HTR2B.

10. The method of claim 1, wherein the therapeutic agent is selected from the group consisting of serotonin receptor inhibitors, serotonin transporter inhibitors, monamine oxidase inhibitors and anti-oxidants.

11. The method of claim 1, wherein the subject does not receive a serotonin release drug.

12. The method of any one of claims 1-11, wherein the subject suffers from the mitral valve disease and receives a mitral valve surgery.

13. The method of claim 12, wherein the mitral valve surgery is selected from the group consisting of mitral valve repair and mitral valve replacement with a prosthesis.

14. The method of any one of claims 1-13, further comprising determining the serotonin transporter polymorphism in the subject.

15. The method of claim 14, wherein the serotonin transporter polymorphism determination comprises performing a genotyping assay on a nucleic acid sample comprising a serotonin transporter gene promoter from the subject.

16. The method of claim 15, wherein the genotyping assay comprises
(a) amplifying a portion of the serotonin transporter gene promoter; and
(b) determining whether the serotonin transporter gene promoter is in an LL form.

17. The method of any one of claims 1-16, wherein the mitral valve disease is a myxomatous mitral valve disease.

18. The method of any one of claims 1-17, further comprising diagnosing the subject as having the mitral valve disease.

19. The method of claim 18, wherein the mitral valve disease diagnosis comprises clinical examination of the subject.

20. The method of claim 18, wherein the mitral valve disease diagnosis comprises documentation of the mitral valve disease in the subject using an imaging technique.

21. The method of claim 20, wherein the imaging technique is selected from the group consisting of cardiac ultrasound, magnetic resonance imaging and cardiac catheterization.

22. The method of any one of claims 1-21, further comprising measuring the blood level of a serotonin transporter gene related biomarker in the subject.

23. The method of claim 22, wherein the biomarker is selected from the group consisting of serotonin, 5-hydroxyindolacetic acid, and a catecholamine.
